(19) United States
(12) Patent Application Publication
(10) Pub. No.: US 2026/0124337 A1
(43) Pub. Date: May 7, 2026

(54) SCENT DIFFUSER DEVICE

(71) Applicant: SCENTIMENT, Inc., Miami, FL (US)

(72) Inventors: Joseph Ayoub, Miami, FL (US); Tamoor Shafi, Miami, FL (US)

(21) Appl. No.: 19/379,293

(22) Filed: Nov. 4, 2025

Related U.S. Application Data
(60) Provisional application No. 63/717,037, filed on Nov. 6, 2024.

(51) Int. Cl. A61L 9/14

(52) U.S. Cl. CPC A61L 9/14; A61L 2209/134

(57) ABSTRACT

A scent diffuser comprises a housing having an open interior, and a top and bottom for closing the open interior, a reservoir container is provided for a scent, a diffuser nozzle, and a pump assembly. The pump assembly has a pump housing and a pump. The pump has a pump air outlet. A pump compartment receives the air pump. The housing includes an access opening and a removable cover for removing and replacing the air pump. An air fitting comprising a socket receives and seals with the pump air outlet. An air conduit communicates between the air fitting and the diffuser nozzle so as to transport air from the pump to the diffuser nozzle. A battery assembly includes a battery and electrical conductors. The electrical conductors communicate with the air pump assembly so as to power the pump. LED lights can communicate diffuser status. A method of operating a scent diffuser is also disclosed.

### CROSS-REFERENCE TO RELATED APPLICATIONS.

This application claims priority to U.S. Provisional Patent Application No. 63/717,037 filed on Nov. 6, 2024, entitled “SCENT DIFFUSER DEVICE”, the entire disclosure of which incorporated herein by reference.

### FIELD OF THE INVENTION

The present invention relates generally to scent diffuser devices.

### BACKGROUND OF THE INVENTION

The use of scent diffuser devices to provide a pleasing aroma in private and public spaces has become prevalent. Such devices in general include a reservoir of aromatic liquid, an air pump, and a diffuser for receiving air from the air pump and liquid from the reservoir and contacting the liquid with the air to atomize the liquid such that the scented air can then be distributed to the surroundings. Devices can come in various shapes and sizes to permit placement in diverse locations and service smaller or larger areas. Over time, the pump can fail due to long-term usage, typically defined by the lifetime hours of the pump. When failure occurs, the entire device must be replaced. Additionally, compact designs are crucial to allow for versatile placement options.

### SUMMARY OF THE INVENTION

A scent diffuser includes a housing having an open interior, and top, bottom and side walls for enclosing the open interior, a reservoir container for a liquid scent, and a nozzle in fluid communication with the reservoir container. The nozzle includes an air nozzle and a liquid nozzle. The air nozzle and liquid nozzle are arranged so as to contact an air stream flowing through the air nozzle with a liquid stream flowing through the liquid nozzle to create an atomized scent stream in the reservoir container. A reservoir outlet is in fluid communication between an atomized scent outlet in the housing and the reservoir container to release the atomized scent stream from the reservoir container into a surrounding environment. An air pump comprises a pump air inlet and a pump air outlet. An air pump compartment can have an open interior dimensioned to receive the air pump and an air fitting making a fluid connection with the pump air outlet of the air pump. An air conduit communicates between the air fitting and the air nozzle. A liquid conduit communicates from the reservoir container to the liquid nozzle. The housing comprises a pump access opening and a detachable access cover for the pump access opening. The pump access opening is dimensioned to permit passage of the air pump for replacing the air pump. A battery assembly comprises a battery and electrical conductors. The electrical conductors are electrically connected with the air pump so as to power the air pump. A switch assembly for controlling the flow of electric current from the battery assembly and operation of the air pump.

The scent diffuser can further include a portable pump cartridge comprising an air pump and structure for engaging the pump compartment. The structure for engaging the pump compartment can be affixed to or part of an exterior of the air pump. The structure for engaging the pump compartment can comprise first and second cartridge portions which when attached to each other define an open interior dimensioned to receive the air pump. The air pump is dimensioned to be received within the open interior of the pump cartridge. The pump cartridge can further comprise first and second electrical contacts extending from an outside surface to an interior surface of the pump cartridge, wherein when the air pump is placed within the pump cartridge electrical contacts on the pump will make electrical contact with the electrical contacts of the pump compartment. One of the first and second electrical contacts on the pump cartridge can be circular and the second of the first and second electrical contacts of the pump cartridge can be inside of the circle. The cover to the access opening can comprise first and second electrical contacts.

The pump air fitting and the pump air outlet can comprise cooperating male and female socket connections. The male socket connection detachably mates with the female socket connection to transfer air from the air pump to the pump air fitting and thereby to the diffuser air nozzle. The female socket connection and the male socket connection permit relative sliding attachment and detachment of the pump air outlet and the air fitting. The pump air fitting can be attached to the pump compartment and can comprise a horizontal nipple and a sealing gasket. The air conduit can comprise a first air conduit extending from the horizontal nipple to a manifold, and a second air conduit extending from the manifold to the diffuser nozzle air inlet.

The scent diffuser can include a status indicator for indicating at least one status characteristic selected from the group consisting of battery life, scent level in the reservoir, and pump operation.

The housing can include a removable diffusion head. The diffusion head can comprise the nozzle. The atomized scent outlet can be provided in the removable diffusion head. A tray can comprise structure for engaging the reservoir container and for engaging the side walls of the housing.

The scent diffuser can include a check valve for preventing fluid flow through the scent outlet opening when the housing is tilted from an upright position. The check valve can incldue a valve member and a valve seat having a valve inlet and a valve outlet. The valve member is movable to close one of the valve inlet and valve outlet to fluid flow when the housing is tilted.

The removable cover can form at least a part of the bottom wall of the housing. The cover can comprise a wireless charging receptor for wirelessly charging the battery.

A method for operating a scent diffuser includes the steps of:
providing a scent diffuser comprising:
   a) a housing having an open interior, and top, bottom and side walls for enclosing the open interior;
   b) a reservoir container for a liquid scent;
   c) a diffuser comprising a diffuser air nozzle and a diffuser liquid nozzle in fluid communication with the reservoir container, the diffuser air nozzle and diffuser liquid nozzle being arranged so as to contact an air stream flowing through the diffuser air nozzle with a liquid stream flowing through the diffuser liquid nozzle to create an atomized air and liquid stream in the reservoir container;
   d) a reservoir outlet in fluid communication between the reservoir container and an atomized air and liquid outlet in the housing to release the atomized air and liquid stream from the reservoir container into a surrounding environment;
   e) an air pump comprising a pump air inlet and a pump air outlet;
   f) a pump compartment having an open interior dimensioned to receive the air pump and an air fitting making a fluid connection with the pump air outlet of the air pump;
   g) an air conduit communicating between the air fitting and the diffuser air nozzle, and a liquid conduit communicating from the reservoir container to the diffuser liquid nozzle;
   h) the housing comprising a pump access opening and a detachable cover for closing the detachable cover, the pump access opening being dimensioned to permit passage of the pump through the pump access opening for replacing the pump;
   i) a battery assembly comprising a battery and electrical conductors, the electrical conductors communicating with the pump assembly so as to power the pump; and,
   j) a switch assembly for controlling the flow of electric current from battery assembly and operation of the pump;
removing the detachable cover;
removing the air pump through the access opening; and,
replacing the air pump with a replacement air pump.

### BRIEF DESCRIPTION OF THE DRAWINGS

There are shown in the drawings embodiments that are presently preferred it being understood that the invention is not limited to the arrangements and instrumentalities shown, wherein:

FIG. 1 is a front perspective view of a scent diffuser according to the invention.

FIG. 2 is a rear perspective view of the scent diffuser of FIG. 1.

FIG. 3 is an exploded perspective view.

FIG. 4 is an exploded perspective view with a reservoir attached to the diffusion head.

FIG. 5 is a bottom exploded perspective view.

FIG. 6 is a bottom exploded perspective view at a greater viewing angle.

FIG. 7 is a front perspective view with the housing removed.

FIG. 8 is a rear perspective view with the housing removed.

FIG. 9 is an exploded perspective with the housing removed and top exploded.

FIG. 10 is an exploded perspective with the housing removed and the top and the bottom exploded.

FIG. 11 is a cross section.

FIG. 12 is an extensively exploded perspective view.

FIG. 13 is an expanded perspective view of area 200 in FIG. 12.

FIG. 14 is an expanded perspective view of area 300 in FIG. 12.

FIG. 15 is an expanded perspective view of area 400 in FIG. 12.

FIG. 16 is an expanded perspective view of area 500 in FIG. 12.

FIG. 17 is a cross section taken along line 17-17 in FIG. 3.

FIG. 18 is an expanded view of area 600 in FIG. 17.

FIG. 19 is a cross section taken along line 19-19 in FIG. 4.

FIG. 20 is an expanded view of area 600 in FIG. 19 and illustrating gas and liquid flows.

FIG. 21 is an expanded perspective view of area 600 in FIG. 19 and illustrating the atomization and discharge of liquid from the scent diffuser.

FIG. 22 is a schematic front elevation of a low output setting and light indicator.

FIG. 23 is a schematic front elevation of a medium output setting and light indicator.

FIG. 24 is a schematic front elevation of a high output setting and light indicator.

FIG. 25 is a bottom perspective view of the scent diffuser in a first stage of pump replacement.

FIG. 26 is an exploded bottom perspective view of the scent diffuser in a second stage of pump replacement.

FIG. 27 is an exploded bottom perspective view of the scent diffuser in a third stage of pump replacement.

FIG. 28 is an exploded bottom perspective view of a pump cartridge in a fourth stage of pump replacement.

FIG. 29 is an exploded top perspective view of a pump cartridge in the fourth stage of pump replacement.

FIG. 30 is an exploded top perspective view of a pump cartridge in the fifth stage of pump replacement.

FIG. 31 is an exploded top perspective view of a pump cartridge in the sixth stage of pump replacement.

FIG. 32 is an exploded bottom perspective view of a pump cartridge in the seventh stage of pump replacement.

FIG. 33 is an exploded bottom perspective of a scent diffuser in an eighth stage of pump replacement.

FIG. 34 is an exploded bottom perspective of a scent diffuser in an ninth stage of pump replacement.

FIG. 35 is bottom perspective view of a sent diffuser in a tenth stage of pump replacement.

FIG. 36 is a bottom perspective view of a first stage of pump replacement according to an alternative embodiment.

FIG. 37 is a bottom perspective view of a second stage of pump replacement according to the alternative embodiment.

### DETAILED DESCRIPTION OF THE INVENTION

A scent diffuser includes a housing having an open interior, and top, bottom and side walls for enclosing the open interior, a reservoir container for a liquid scent, a diffuser nozzle in fluid communication with the reservoir container and including an air nozzle and a liquid nozzle. The air nozzle and liquid nozzle are arranged so as to contact an air stream flowing through the air nozzle with a liquid stream flowing through the liquid nozzle to create an atomized liquid scent stream in the reservoir container. A reservoir outlet is in fluid communication between an atomized liquid scent outlet in the housing and the reservoir container to release the atomized liquid stream from the reservoir container into a surrounding environment. An air pump includes a pump air inlet and a pump air outlet. An air pump compartment has an open interior dimensioned to receive the air pump and an air fitting making a fluid connection with the pump air outlet of the air pump. An air conduit communicates between the air fitting and the air nozzle. A liquid conduit communicates from the reservoir container to the liquid nozzle.

The housing comprises a pump access opening and a detachable access cover for the pump access opening. The pump access opening is dimensioned to permit passage of the air pump for replacing the air pump. The access opening and access cover are provided at the bottom of the housing, but could alternatively be located elsewhere.

A battery assembly includes a battery, a battery holder, and electrical conductors. The electrical conductors are electrically connected with the air pump so as to power the air pump. A switch assembly is provided for controlling the flow of electric current from the battery assembly and operation of the air pump. The access cover can comprise a wireless charging receptor for wirelessly charging the battery. The housing can include a wired charging port such as USB-A or USB-C.

The scent diffuser can include a portable pump cartridge comprising the air pump and an outer cartridge shell for containing the pump and for engaging the pump compartment. The shell can be dimensioned to fit tightly within the pump compartment. Alternatively, the pump and the pump cartridge can be dimensioned to fit together such that a separate pump cartridge and shell are not necessary. The pump cartridge can comprise first and second cartridge portions which when attached to each other define an open interior dimensioned to receive the air pump, and wherein the air pump is dimensioned to be received within the open interior of the pump cartridge shell. The pump cartridge can further comprise first and second electrical contacts extending from an outside surface to an interior surface of the pump cartridge shell, wherein when the air pump is placed within the pump cartridge shell electrical contacts on the pump will make electrical contact with the electrical contacts of the pump cartridge shell. The first and second electrical contacts on the outside of the pump cartridge shell can be circular and the second of the first and second electrical contacts of the pump cartridge can be inside of the circle. The cover to the access opening can include first and second electrical contacts.

The pump air fitting associated with the pump compartment and the pump air outlet of the air pump can comprise cooperating male and female socket connections. Other connection structure is possible. The male socket connection detachably mates with the female socket connection to transfer air from the air pump to the pump air fitting and air conduit and thereby to the diffuser air nozzle. The female socket connection and the male socket connection permit relative sliding attachment and detachment of the pump air outlet and the air fitting.

The air fitting can be attached to the pump compartment and comprises a horizontal opening and a sealing gasket. An air conduit comprises a first air conduit extending from the air fitting to a manifold, and a second air conduit extending from the manifold to the diffuser nozzle air inlet.

The scent diffuser can include a status indicator for indicating at least one status characteristic selected from the group consisting of battery life, scent level in the reservoir, and pump operation.

The atomized scent outlet can be provided in a removable diffusion head or cap. The diffusion head can comprise structure for engaging the reservoir container and for engaging with the housing. Magnets can be provided to facilitate this engagement. Other engagement structure is possible. A check valve can be provided in the diffusion head for preventing fluid flow through the scent outlet opening when the housing is tilted from an upright position. The check valve comprises a valve member and a valve seat having a valve inlet and a valve outlet. The valve member, which can be a metal sphere, is movable to close one of the valve inlet and valve outlet to fluid flow when the housing is tilted.

A method for operating a scent diffuser, comprising the steps of: providing a scent diffuser comprising a housing having an open interior and top, bottom and side walls for enclosing the open interior, a reservoir container for a liquid scent, and a diffuser nozzle having a diffuser air nozzle and a diffuser liquid nozzle in fluid communication with the reservoir container. The diffuser air nozzle and diffuser liquid nozzle are arranged so as to contact an air stream flowing through the diffuser air nozzle with a liquid stream flowing through the diffuser liquid nozzle to create an atomized air and liquid stream in the reservoir container. A reservoir outlet is in fluid communication between the reservoir container and an atomized air and liquid outlet in the diffusion head or the housing to release the atomized air and liquid stream from the reservoir container into a surrounding environment. An air pump includes a pump air inlet and a pump air outlet. A pump compartment has an open interior dimensioned to receive the air pump and an air fitting making a fluid connection with the pump air outlet of the air pump. An air conduit communicates between the air fitting and the diffuser air nozzle, and a liquid conduit communicates from the reservoir container to the diffuser liquid nozzle. The housing comprises a pump access opening and a detachable cover for closing the detachable cover. The pump access opening is dimensioned to permit passage of the pump through the pump access opening for replacing the pump. A battery assembly comprises a battery and electrical conductors, the electrical conductors communicating with the pump assembly so as to power the pump. A switch assembly is provided for controlling the flow of electric current from battery assembly and operation of the pump. The method includes removing the detachable access cover. The air pump is then removed through the access opening. A replacement air pump is then passed though the access opening, and the access cover secured to close the access opening.

The access cover can be provided at the bottom, sides or top of the housing. In one embodiment, the access cover forms a detachable bottom and the pump is removed and replaced through a bottom opening into the pump cartridge compartment. The access opening and access cover, and the pump, can be provided in different positions relative to the housing, such as the sides or the top.

The manner of detachment of the access cover to the housing can vary. Screws, hinges, latches, straps, snaps, threads, cooperating press fit sections, and many other constructions are possible for detaching the cover from a position closing the access opening.

The pump air outlet and the air fitting associated with the pump compartment can take many forms. In one embodiment, they have a plug and socket or male/female configuration in which the pump air outlet is in the form of a plug and the air fitting is in the form of a socket. The plug of the pump will disengage from the socket of the air fitting when the pump is removed from the housing. Many other configurations are possible to form a detachable and hermetic seal between the pump air outlet and the air conduit inlet.

There is shown in the figures a scent diffuser 10 having a housing 14 with an open interior 16 and an open bottom 18 and open top 24. A base 28 to stabilize the scent diffuser and can define an access opening 34. A bottom access cover 30 detachably secures to the base 28 and closes the access opening 34. A removable top cap or diffusion head 32 can be provided to close the open top 24 and can have an atomized scent outlet 33. A decorative top 78 can be applied and has an opening 79 aligned with the outlet opening 33. A pump cartridge 40 can comprise a pump cover or shell which can enclose the pump 45. The pump cartridge can be one piece of more pieces such as first cartridge shell portion 42 and second cartridge shell portion 44. The pump has pump air inlets 47 and a pump air outlet 48 which can extend through a suitable opening 49 in the first cartridge shell portion 42.

A liquid reservoir container 50 is provided for the scent oil or other liquid. The liquid reservoir container 50 can be attached to the diffusion head 32 by suitable structure such as cooperating threads 51 on the reservoir container 50 and threads 52 fixed to the diffusion head 32. A dip tube or conduit 54 extends into the reservoir container 50 to transport scent oil liquid from the reservoir container 50 to a nozzle 55.

A pump compartment 60 is provided for receiving the pump cartridge 40, or in some embodiments a standalone pump that is not part of a cartridge. The pump compartment 60 has an open interior 64 which receives the pump cartridge 40. The pump cartridge 40 can thereby be placed through the open bottom 18 and into the pump compartment 60. An air outlet 68 which can be in the form of a socket in the pump compartment 60 receives the pump air outlet 48 of the pump 45. The access cover 30 can be used to also close the opening 64 to secure the pump cartridge 40 within the pump compartment 60.

The air outlet 68 communicates with an air conduit which delivers air to the diffuser nozzle 55. The air conduit can be a single piece or double pieces such as a first air conduit section 70 and a second air conduit section 74 which can be joined by a manifold connector 440, which allows for connection of both at or near the base 28 and facilitates assembly and disassembly by placing both connections near an opening of the housing 14.

A battery 80 can be provided to power the pump 45 and other electronic components. The battery 80 can be comprised of cells 81 and 82 or any number of battery cells. The battery 80 can be secured in place by a battery housing 83. Receptacles 93 on the access cover 30 receive pins 95 on the housing 14 to transfer power from the battery to the pump 45. A front light printed circuit board assembly (PCBA) 110 supports an LED lights 115 and light diffuser 114. The light emanating from the light diffuser 114 is visible through an opening 116 in the housing 14. Electrical connections 118 receive signals and power to cause the LEDs 115 to indicate the appropriate amount, color and flashing sequence of light to indicate various diffuser settings and operational modes. The front light printed circuit board assembly (PCBA) 110 can be held in place by suitable fasteners such as screws 117.

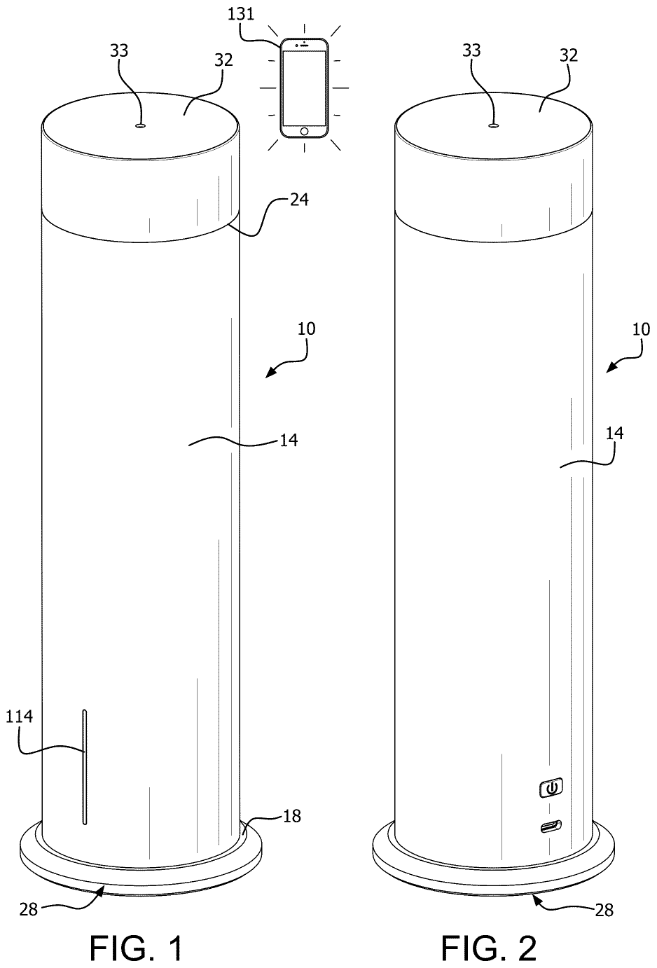

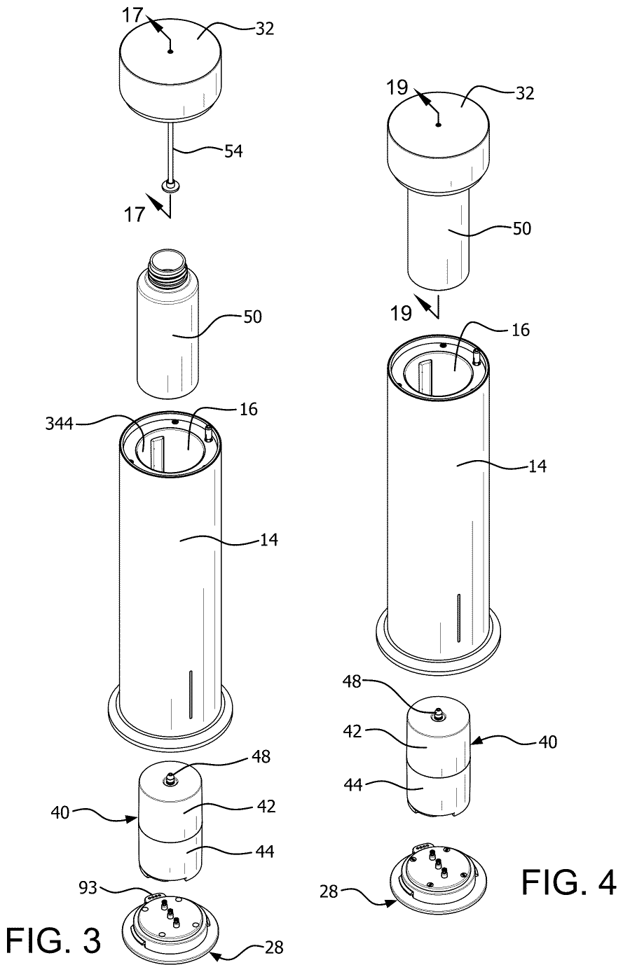

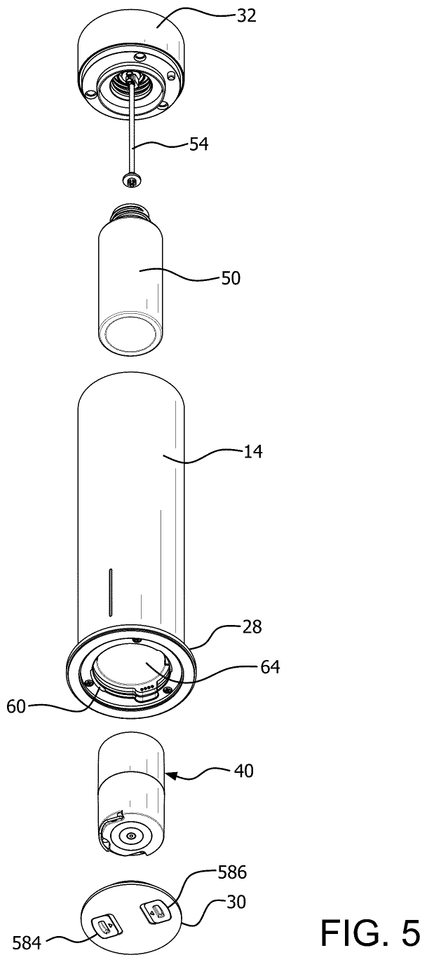

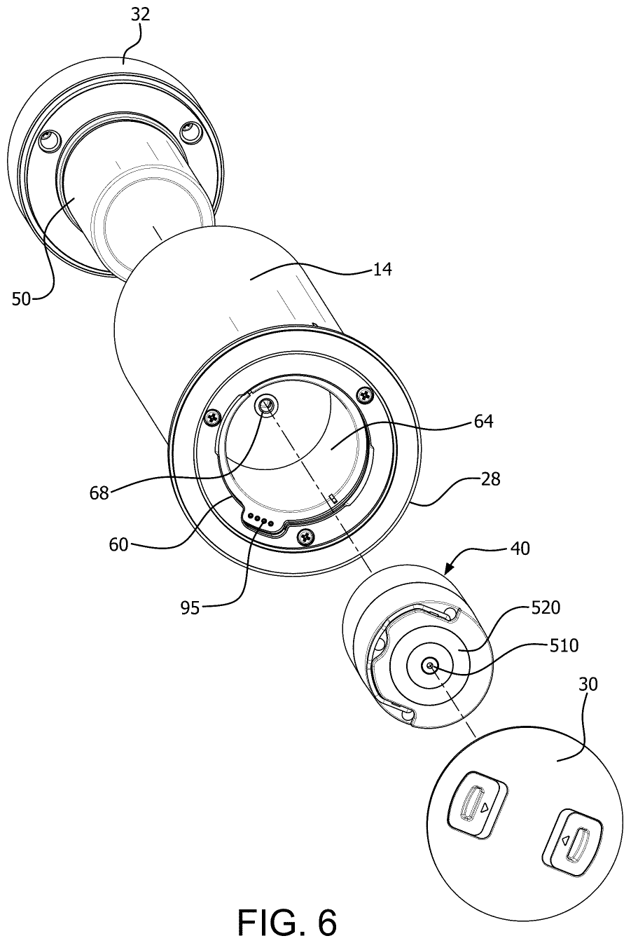

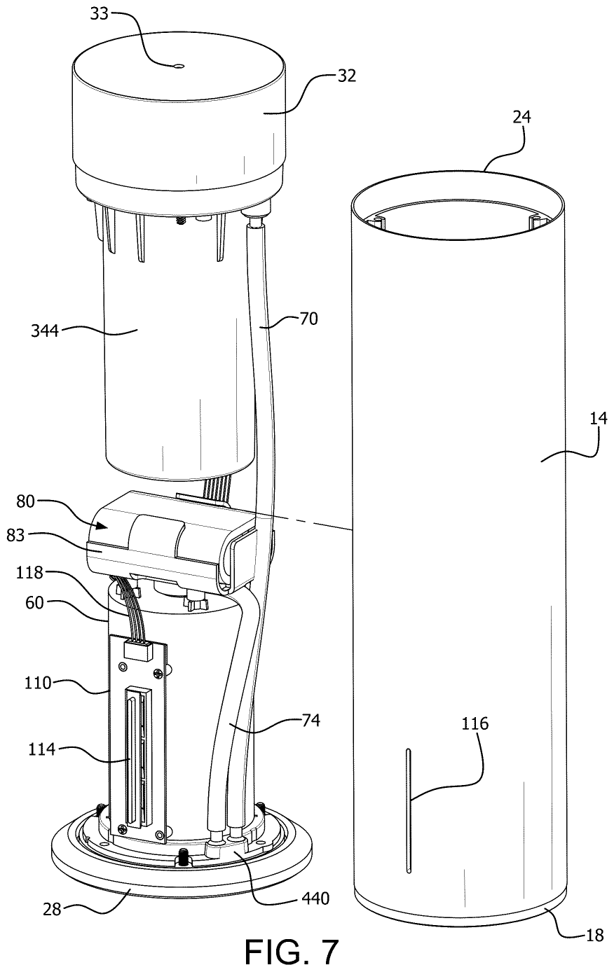

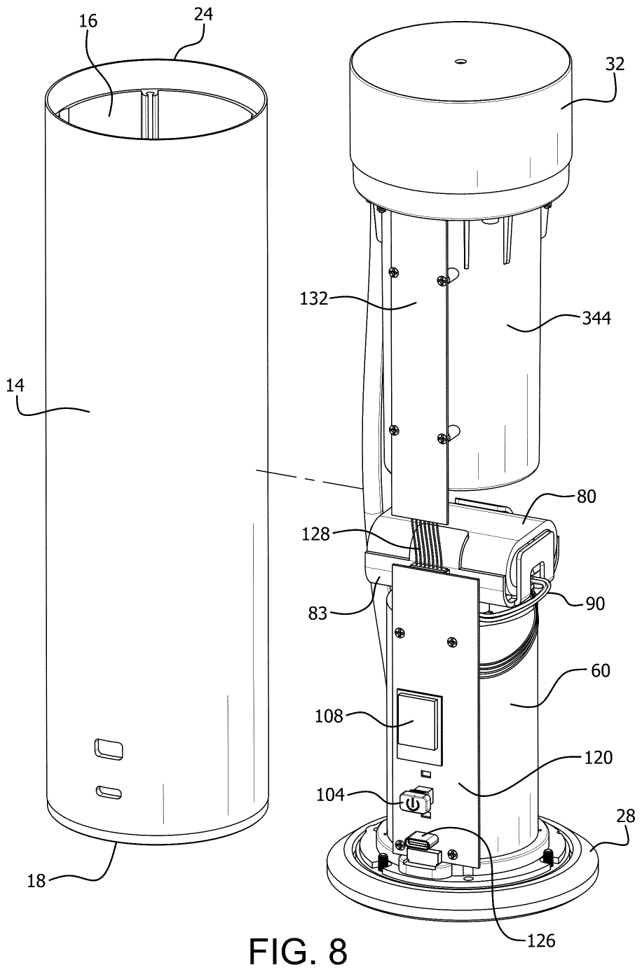

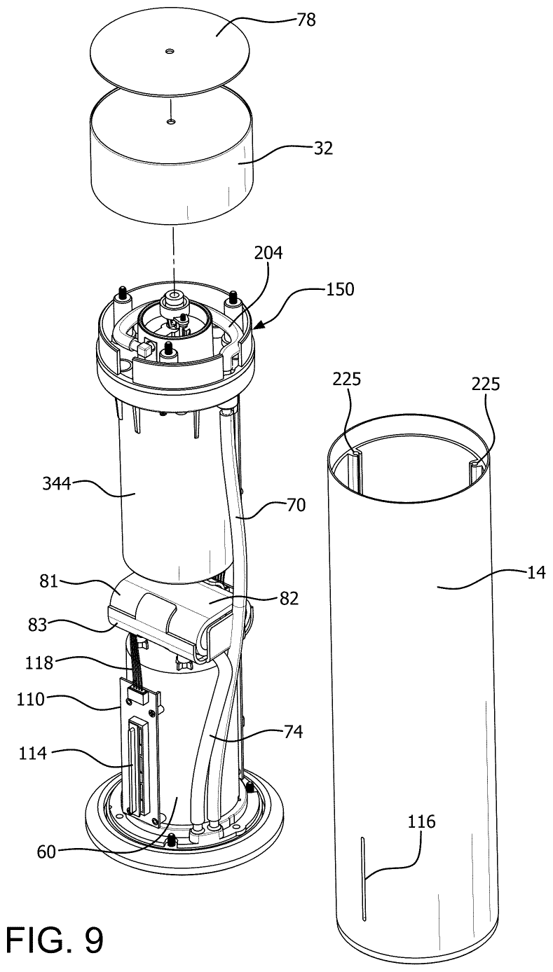

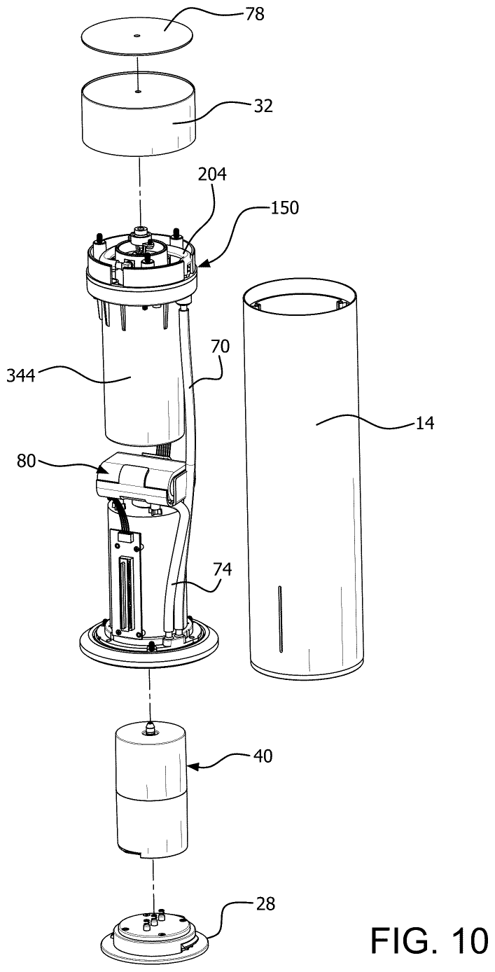

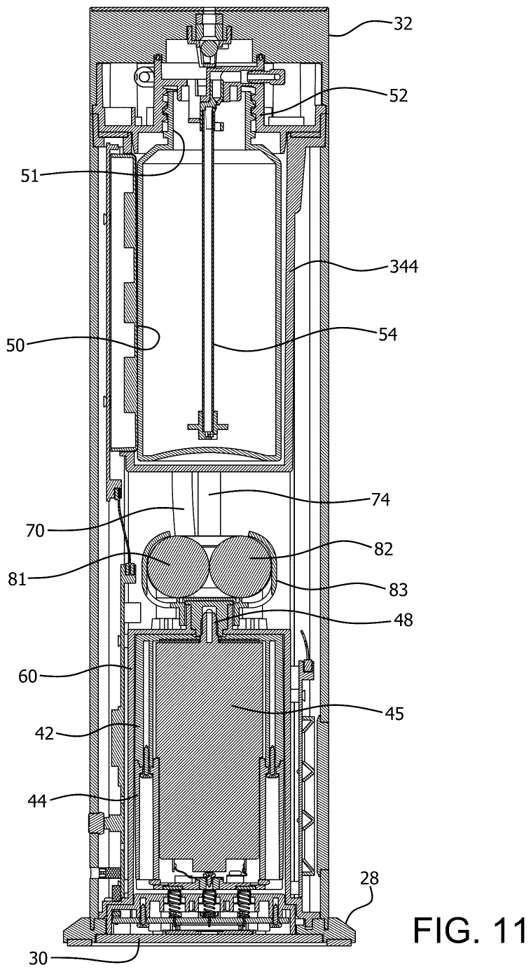

Electrical connections 90 deliver power from the battery 80 through a power switch button 104 mounted on a main PCBA 120 to power the pump 45 and various operations of the main PCBA 120. A WiFi chip 108 enables wireless communication between the diffuser 10 and a mobile application such as a remote or a mobile communications device or cellphone 131 (FIG. 1), by leveraging a WiFi connection. The wireless communication allows the user to manage the operation of the device such as but not limited to turning the device ON and OFF, increasing or decreasing the mixing intensity. Electrical connections 128 communicate between the main PCBA 120 and a scent liquid level sensor PCBA 132 that supports and operates a scent liquid level sensor 340.

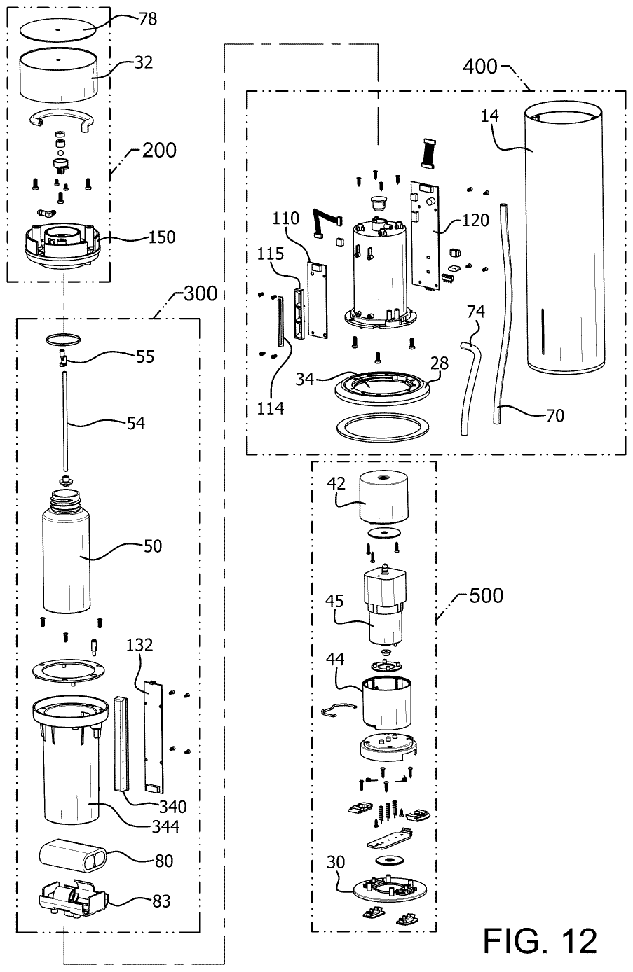

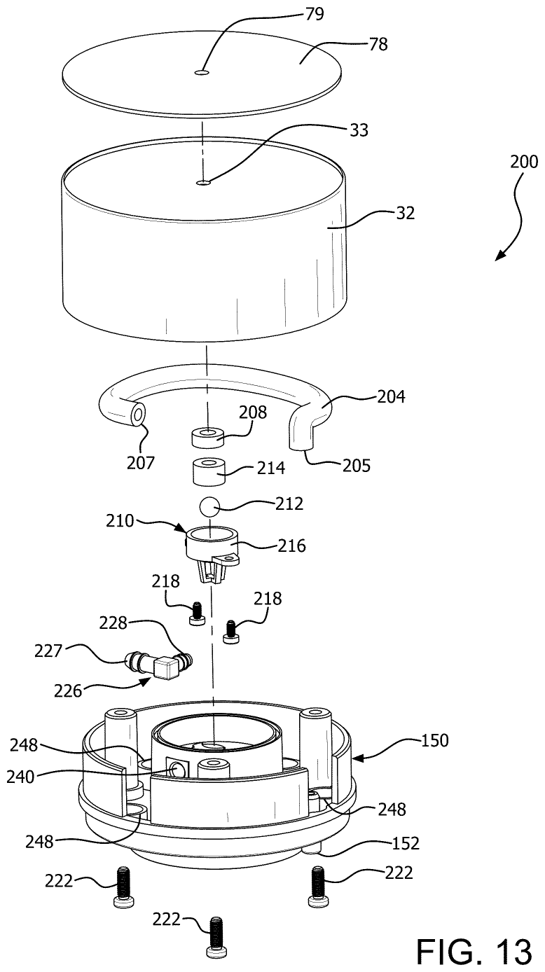

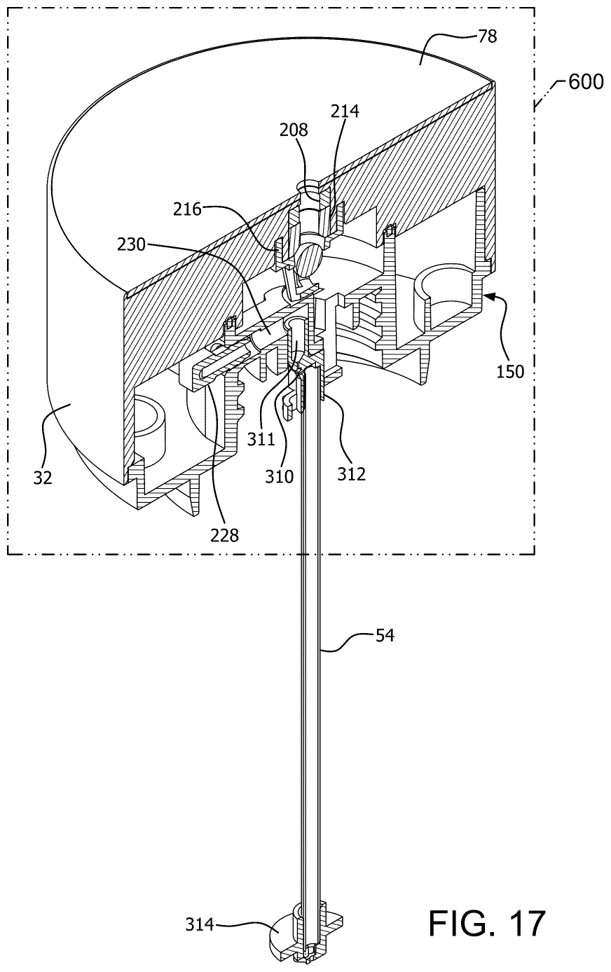

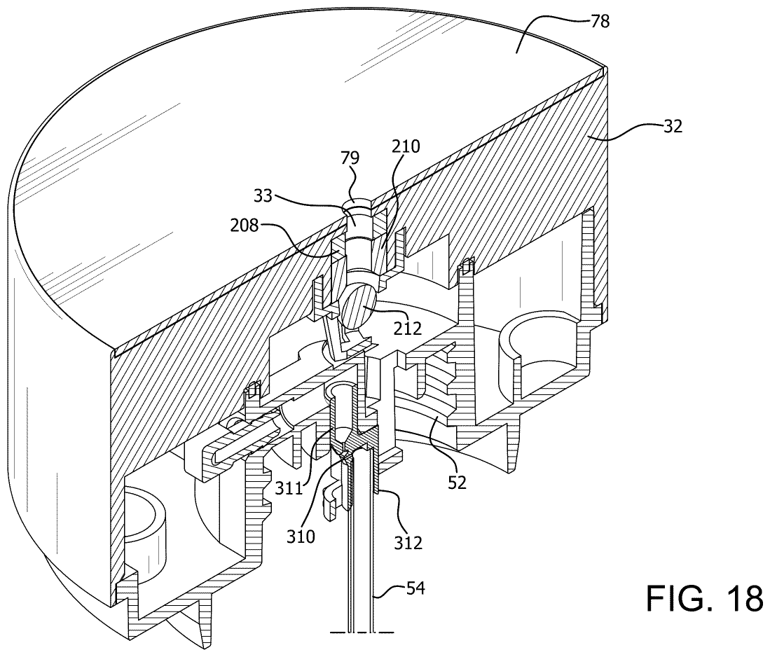

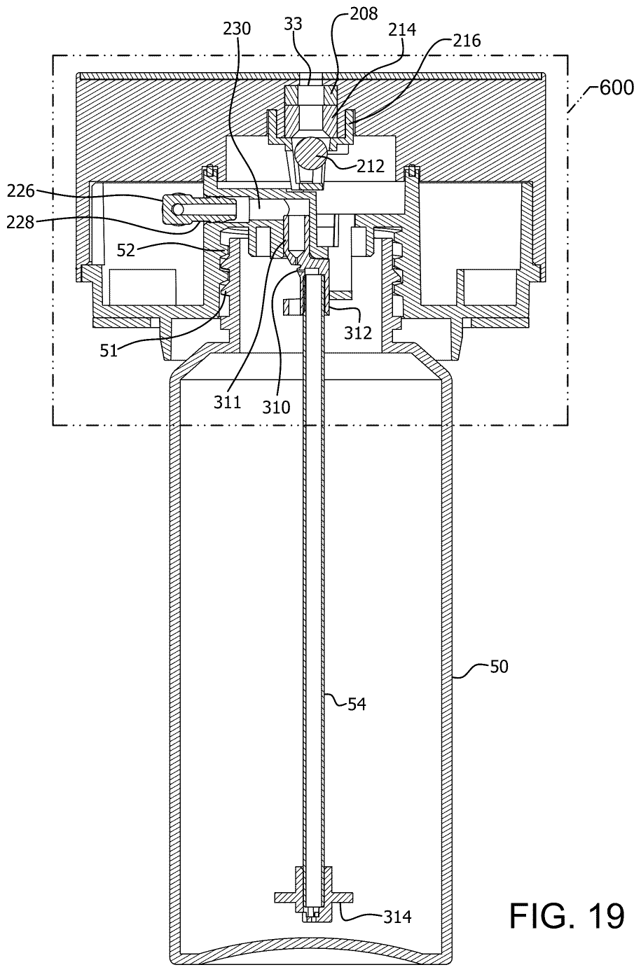

There is shown in FIGS. 12-21 views of the scent diffuser 10 according to the invention. The components can be segregated for ease of understanding into diffusion head area or section 200, liquid scent compartment section 300, housing section 400, and pump cartridge and access cover section 500. As shown in FIG. 13, the diffusion head section 200 includes the diffusion head 32 and atomized scent outlet opening 33, and a diffusion head tray 150. A fitting 152 in the diffusion head tray 150 connects to a fitting 155 that is connected to the housing 14. The fitting 155 connects to the first air conduit section 70 and thereby receives air from the pump 45. An air hose 204 connects between the fitting 152 and the diffuser nozzle 55. The air passes through end 207 of the air hose 204 to air connector fitting 226 with a passageway 231. The air connector fitting 226 has an air inlet 227 and an air outlet 228 and acts as a point of connection for the air to the nozzle 55 through opening 240.

A check valve 210 prevents unwanted leaks if the scent diffuser 10 is tipped or tilted. A steel sphere 212 is provided to seal the outlet 33 when the diffuser is tipped or tilted. A strong magnet 208 keeps the anti-leak steel sphere 212 in place when the diffuser is tipped or tilted. A silicone rubber seal 214 is contacted by the steel sphere 212 to form a seal when the diffuser 10 is tipped or tilted to prevent leaks. The spherical steel sphere 212 acts as a barrier for the escape of liquid from the reservoir. A one-way valve housing 216 is the main housing for the anti-leak system comprising the steel sphere 212. Fasteners such as screws 218 hold the anti-leak valve housing 216 in place inside the diffusion head 32.

Diffusion head internal screws 222 secure the diffusion head tray 150 to the diffusion head 32. An intake air conduit 230 is an embedded conduit that channels the air from the air hose 204 into the nozzle 55. Magnets 248 in the diffusion head tray 150 allow the diffusion head 200 to remain in place when installed on the scent diffuser 10.

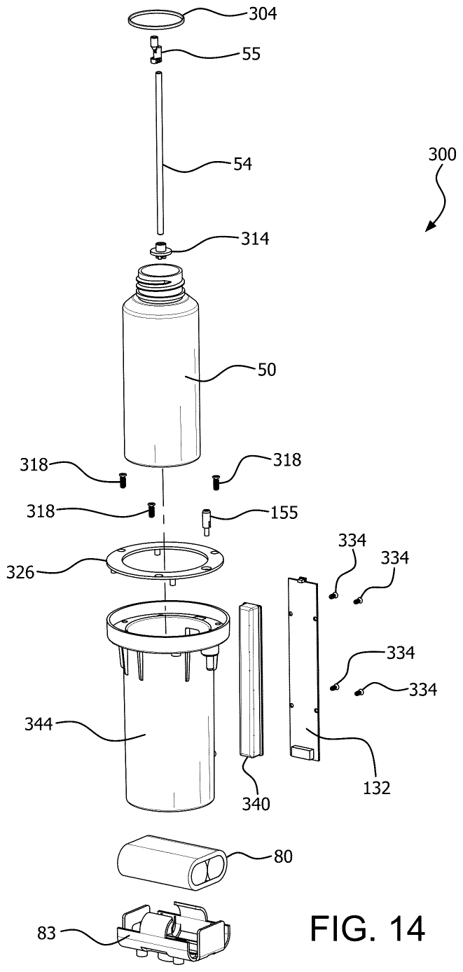

The scent liquid compartment area 300 is shown in FIG. 14. A gasket 304 helps to secure the reservoir container 50 when installed in the diffusion head 200. The diffusion nozzle 55 atomizes the fragrance oil by leveraging Bernoulli's principle. The dip tube 54 is used to withdraw scent oil from the reservoir container 50 and connects to oil inlet 312 of the nozzle 55. The scent oil exits an oil nozzle 310 and the air exits an air nozzle 311. The two streams contact one another to atomize the oil in the air. The reservoir container 50 serves as a reservoir for the scent oil or other scent liquid. A scent liquid level indicator 314 slides up and down over the dip tube 54 to provide an indication of oil level in the reservoir container 50. A reservoir compartment 344 can be provided to receive the reservoir container 50. Reservoir compartment screws 318 secure the reservoir compartment 344 to the housing 14. A connector 320 connects with the first air conduit 70 and also the fitting 152 from the diffusion head 200 to allow the air to flow from the air pump 40 to the diffusion head 200. The fitting 155 also acts an alignment indicator when placing the diffusion head 32 in place on the housing 14, because the lower end of the fitting 152 must be received in an upper female portion of the fitting 155 and the user can sense when this sliding engagement has occurred. The fitting 155 can be secured to a metallic ring 326 that is fixed to a top portion of the housing 14. The metallic ring 326 on the housing 14 allows magnets 248 on the diffusion head 32 to keep it in position when placed on the housing 14.

Oil sensor PCBA screws 334 keep the oil sensor board 110 in place. The oil level sensor 340 provides oil level readings based upon the position of the scent liquid level indicator 314 in the reservoir container 50 by using optical or magnetic sensors. The reservoir compartment 344 will keep the reservoir container 50 protected when inside the diffuser 10 and will allow the oil level sensor 342 to monitor the oil level.

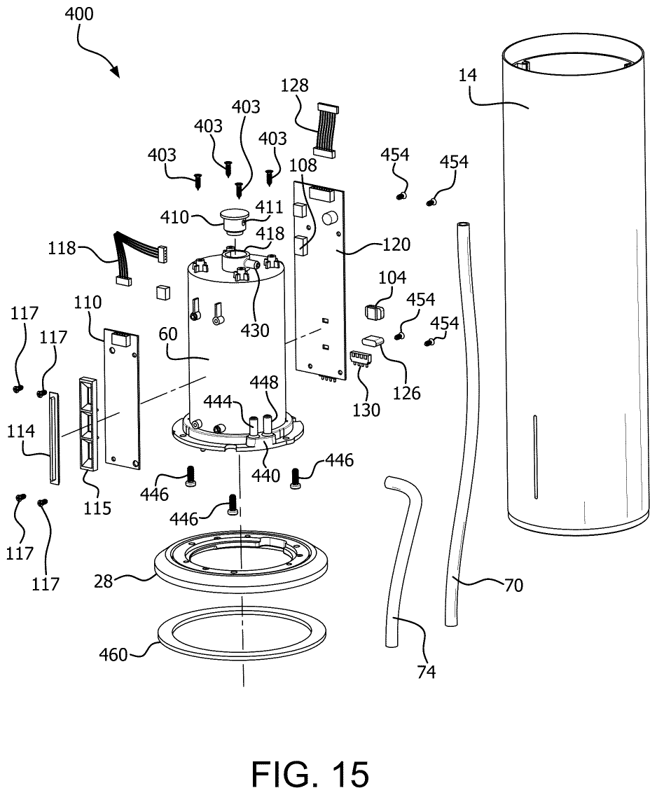

The housing section 400 is shown in FIG. 15. The external body or housing 14 can be made of an aluminum alloy or other suitable material. The main PCBA 120 is secured to the pump compartment 60 by main PCBA screws 454. The main PCBA 120 is the main computing board that controls all other PCBA's and the diffuser functions. The WiFi chip 108 can be used to provide communication with the main PCBA 120 from a mobile communications device or cellphone. The power and data cable 128 is the bridge of power and data communication between the oil sensor PCBA and the main PCBA 120. The main PCBA 120 wireless power connection 130 is the point of connection with the wireless power receptor main PCBA 120. The power switch button 104 is used to turn the diffuser on and off. Battery housing screws 403 are designed to attach the battery to the pump compartment 60. The USB type C power port 126 is designed to receive the charging cable.

Rubber gasket 410 is a point of connection for the pump air outlet 48 to the air outlet 68. The rubber gasket 410 allows a change of direction for the air to transition into a horizontal level through opening 411. The front light data and electrical connection power wires 118 are a bridge of power and data communication between the front light bar (intensity indicator) PCBA 110 and the main PCBA 120. Light bar PCBA screws 117 are designed to retain the light bar PCBA 110 on the pump compartment 60. Light diffuser 114 provides light alerts to communicate to the user the light intensity levels, oil level alerts and battery alerts. The light diffuser 114 makes the light coming from the LEDs look even more visible and appealing. LED lights 115 provide the actual light signals. The rubber gasket 410 fits into a receptacle air fitting 418 which has a horizontal nipple 430 for connecting to the second air conduit section 74. A manifold connector 440 has an inlet 444 for receiving the second air conduit section 74 and an outlet 448 for receiving the first air conduit section 70. Base screws 446 secure the diffuser base 28 to the housing 14. A rubber pad 460 prevents the diffuser from sliding on surfaces.

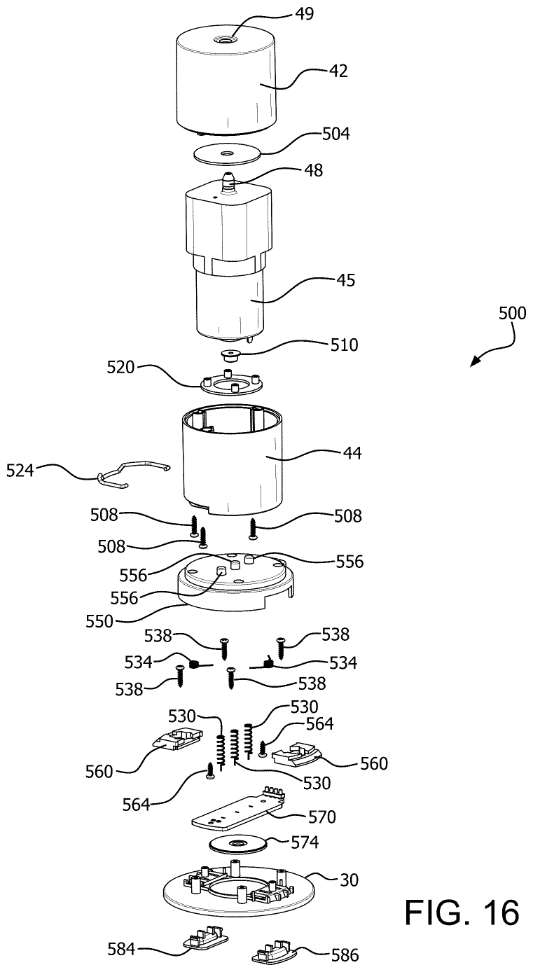

The pump cartridge section 500 is shown in FIG. 16. The pump shell top 42 and bottom 44 house the pump 45. A cushion pad 504 is designed to reduce vibrations and noise from the pump 45. Pump cartridge screws 508 secure both the top and bottom pump cartridge shells 42 and 44 together. The pump air nipple 48 acts as the air exit point from the pump 45. The air pump 45 is the component that generates the airflow that produces the atomized fragrance. A conductive copper rivet center electrode 510 acts as the conductor for the opposite charge from the conductive ring electrode 520. The conductive ring electrode 520 is designed to transfer the electric charge to the air pump 45. A pump cartridge handle 524 is a metallic handle designed to allow the user to pull the pump cartridge 40 out of the pump compartment 60 from the bottom. Ejection springs 530 push the access cover 30 out when the locking mechanism is released, and help to transmit power from the wireless charging PCBA 570 to the conductive ejection barrels 556. Bottom internal cover screws 538 are designed to secure the internal plastic cover to the bottom gate 30. A bottom gate cover 550 protects the wireless charging PCBA 570 and the locking mechanism. Conductive ejection barrels 556 have two functions. They are part of the ejection system that pushes the access cover 30 out when the locking mechanism is released. They also act as power conductors between the wireless charging PCBA 570 and the copper rivet center electrode 510 and conductive ring electrode 520 on the bottom of the pump cartridge. Locking mechanisms 560 are plastic pieces that make the bottom access cover locking mechanism to keep the cover in place when installed back on the diffuser. Torsion springs 534 allow the locking mechanisms 560 to retract when released. Wireless charging PCBA screws 564 are meant to keep the wireless charging PCBA attached to the bottom gate internal cover. Wireless charging PCBA 570 is a computing board to control the wireless charging feature. Wireless charging receptor 574 is a metallic coin designed to receive the wireless power from the charger and transmit it to the wireless charging PCBA. The bottom gate 30 is released to allow access to the air pump 45. Locking mechanism sliders 584 and 586 are the sliding parts of the locking mechanism. These will be the moving piece when the user pinches to release the gate 30.

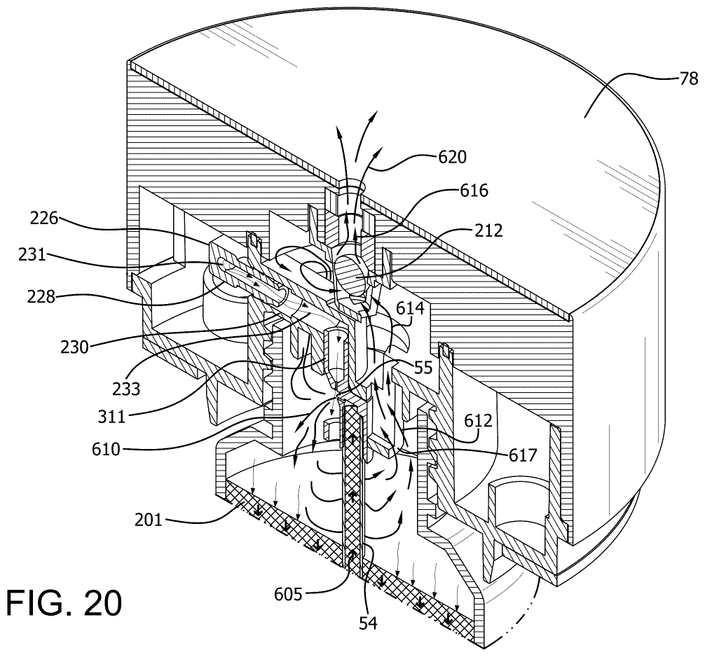

There is shown in FIGS. 17-21 the operation of the nozzle 55. The dip tube 54 is immersed in the scent oil in the reservoir container 50. As the pump 45 operates, the air flows through the conduits 70 and 74 direct air to the air outlet 228 of the air connector fitting 226 as stream 231here it enters intake air conduit 230 as an air stream 233 (FIG. 20). The air stream 233 passing through intake air conduit 230 passes through into the air nozzle 311 of the diffuser nozzle 55. Atomized scent flow 610 puts pressure on the scent oil 201 in the reservoir container 50, forcing scent oil shown by arrow 605 up the dip tube 54. The air flow 612 over the oil 201 circulates upward and flows out of reservoir outlet 617 as an atomized stream 614 past the sphere 212 as an atomized stream 616 and exits as an atomized stream 620.

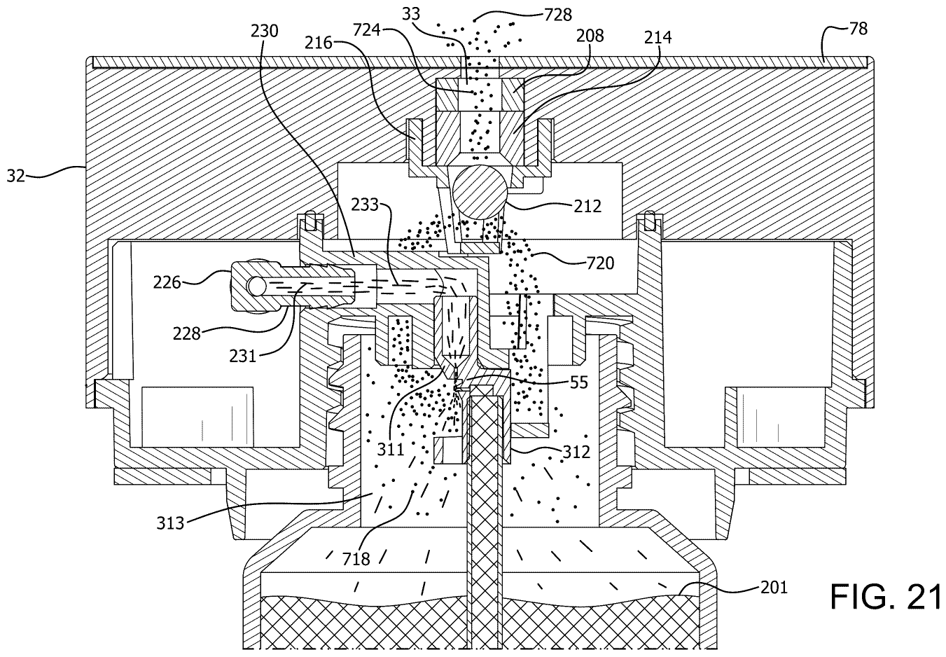

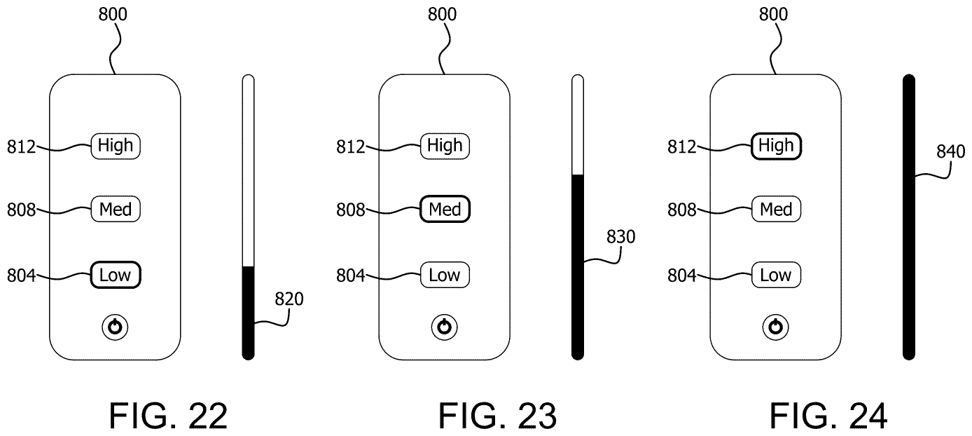

Atomization of the scent oil occurs at the nozzle 55 as shown in FIG. 21. Oil passes through oil inlet 312 of the diffuser nozzle 55 and strikes the air emanating from the air nozzle 311 to atomize the oil into micro droplets 718. Air pressure within the vapor space 313 above the oil 200 applies pressure to the oil to force the oil upward through the dip tube 54 as shown by arrow 605. The oil micro droplets 718 in the vapor space 313 flow upward as rising micro droplets 720 and pass the metal sphere 212. Atomized droplets 724 pass through the channel 33 and leave as dispersed micro droplets 728.

The device emits fragrance into the air with the support of the pump which operates through a cyclic process, controlled by consumer-selectable settings. Each cycle consists of an Activation Phase, where the pump turns on for a predetermined duration atomizing fragrance, followed by an Idle Phase, where the pump turns off allowing fragrance dissipation. Cycle duration combines Activation and Idle Phases, while Motor Lifetime accumulates Activation Phase durations. Pump specifications vary based on coverage area, with pumps tailored for specific ranges. Control settings adjust fragrance emission frequency and/or duration: Hi (increased), Med (moderate), and Low (decreased), enabling customizable fragrance intensity.

The diffuser 10 can have different settings affecting the pump 45. The device emits fragrance into the air through a cyclic process, controlled by consumer selectable settings. Each cycle consists of an Activation Phase, where the pump turns on for a predetermined duration atomizing fragrance, followed by an Idle Phase, where the pump turns off allowing fragrance dissipation. Cycle duration combines Activation and Idle Phases, while Motor Lifetime accumulates Activation Phase durations. Device specifications vary based on coverage area, with pumps tailored for specific ranges. Control settings allow the consumer to customize their scenting intensity. The LED lights 115 can show the pump setting. For example, a low setting obtained by pressing button 804 on panel 800 can lead to a low light signal 820, for example by illumination of a single LED light. A medium setting by button 808 can result in a larger light signal 830 as by two LED lights. A high setting button 812 can result in all three lights being activated 840 to indicate maximum power. The lights can also be used to indicate battery level and oil level. Different indications can be provided by differently colored LED lights. Oil levels can be determined by the scent oil liquid level indicator 314 and scent oil liquid level sensor 340, depending on the level of oil in the container 50. The position read by the oil level sensor 340 and communicated to the scent liquid level sensor PCBA 132 and thereby to the main PCBA 120 and ultimately to the LED light PCBA 110, and LED lights 115.

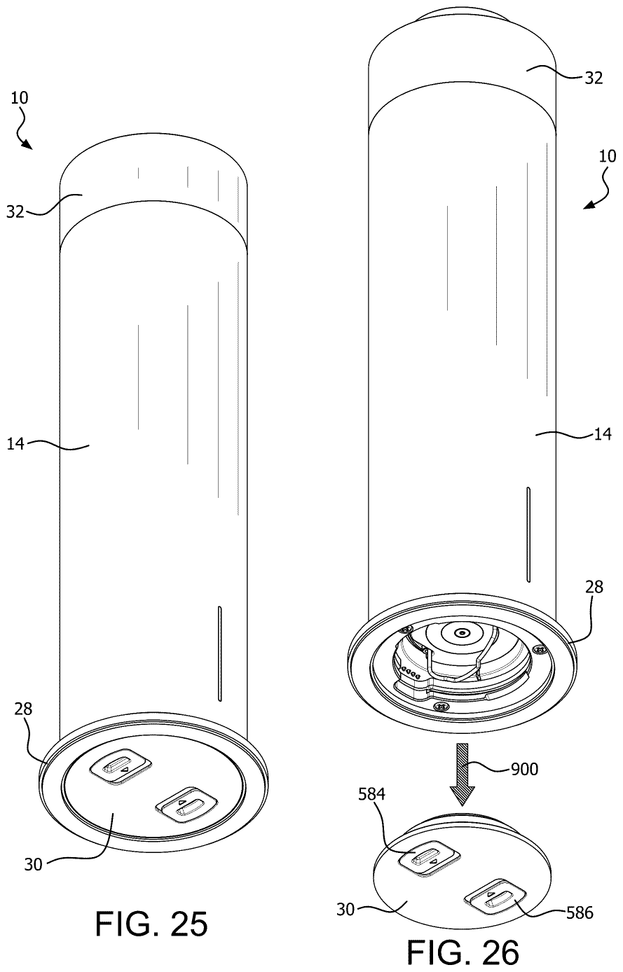

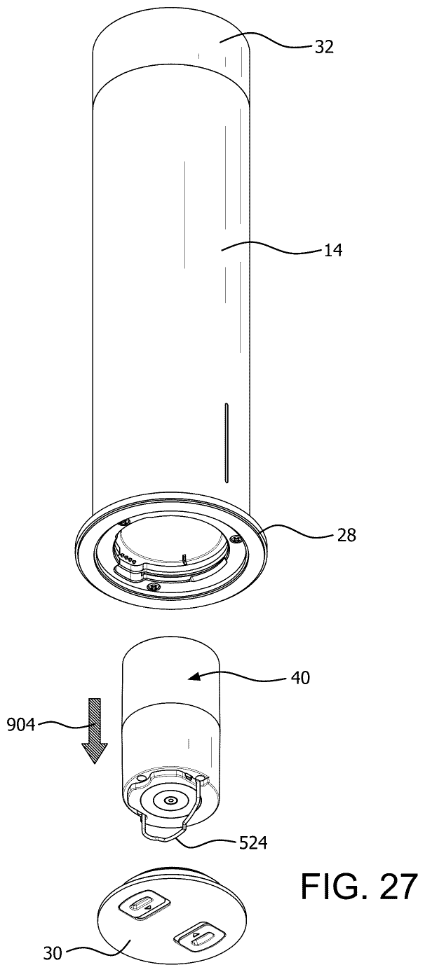

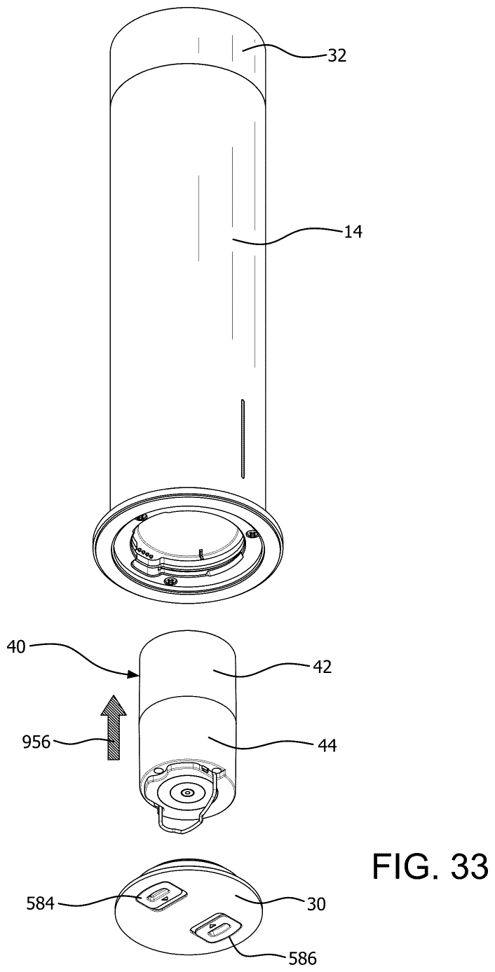

There is shown in FIGS. 25-35 a method for changing a pump in a scent diffuser 10 according to the invention (FIG. 25). This can occur for example when a pump fails. Normally, the user would need to replace the entire scent diffuser 10. The user can remove the access cover 30 (FIG. 26). The user can then pull out the pump cartridge 40 from the housing 14 using the pump cartridge handle 524 (FIG. 27). At this point, the user would simply replace the cartridge with another cartridge 40 and return the access cover 30 to its position. The pump air outlet 48 is guided into the air inlet fitting 418 and rubber gasket 410 by the act of insertion, because the pump compartment 60 aligns and guides the pump cartridge correctly to mate the pump air outlet 48 with the rubber gasket 410 and air inlet fitting 418 associated with the pump compartment 60 (FIG. 33).

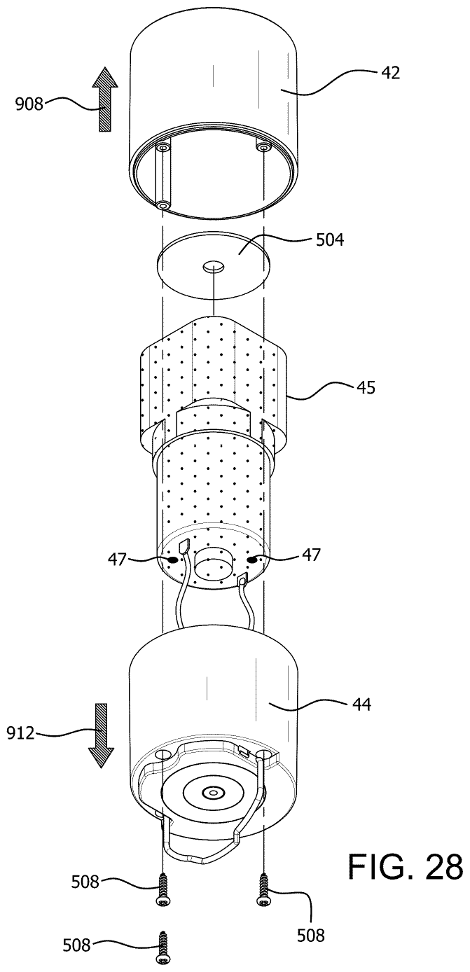

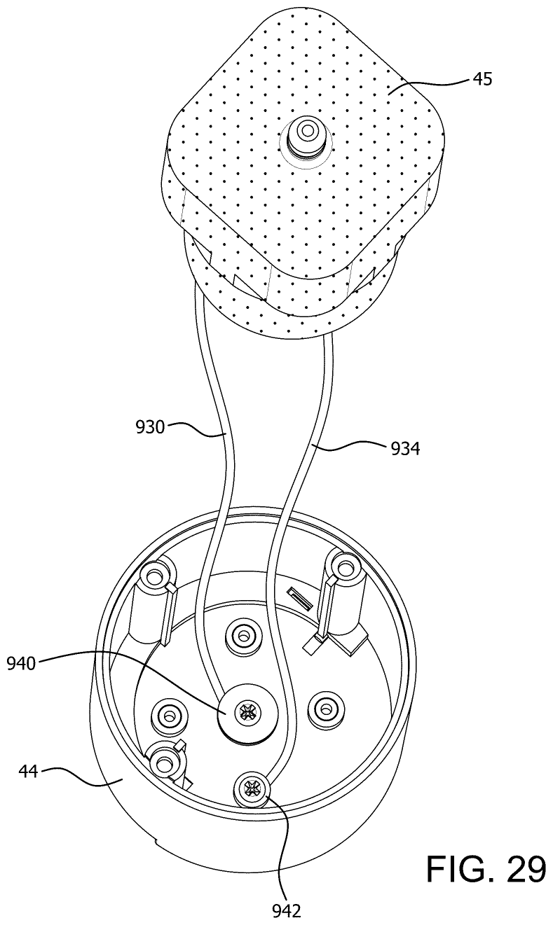

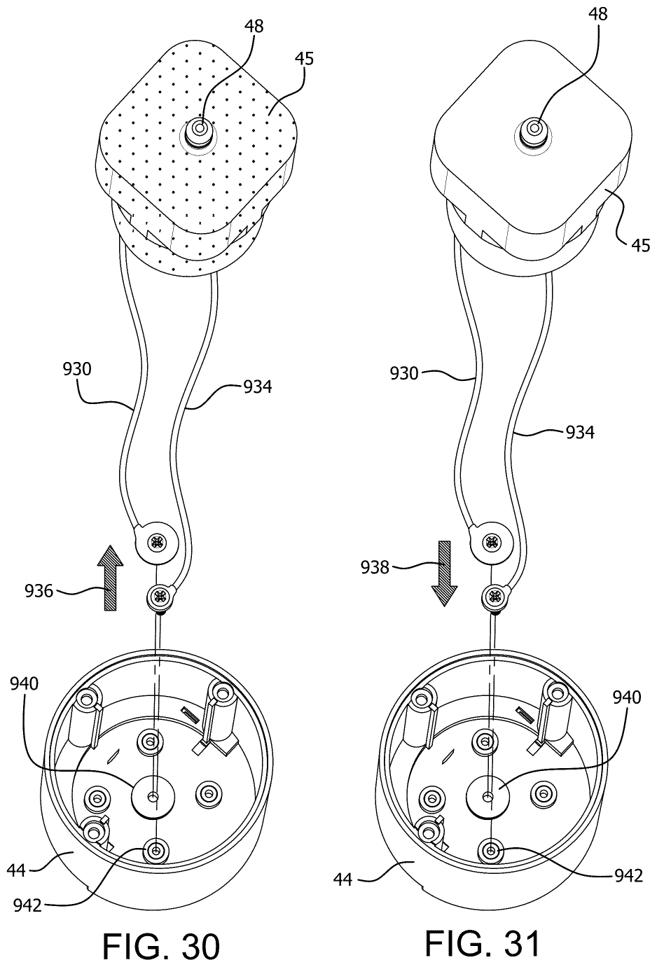

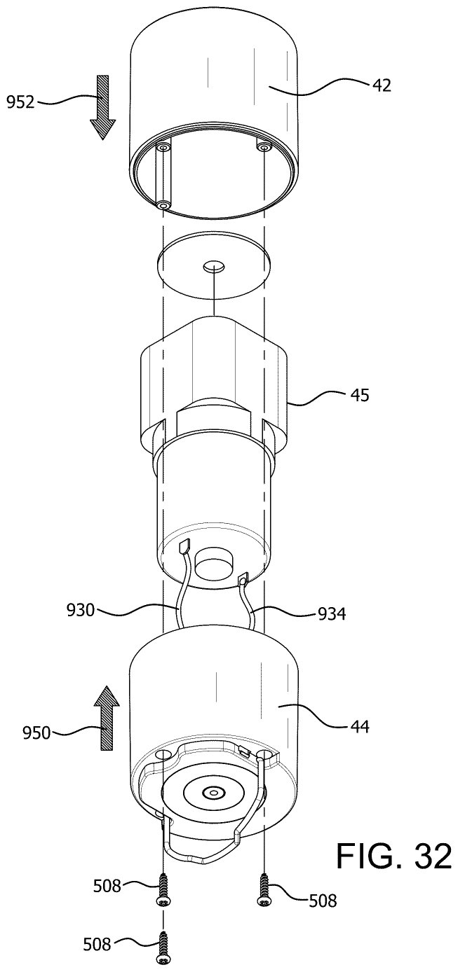

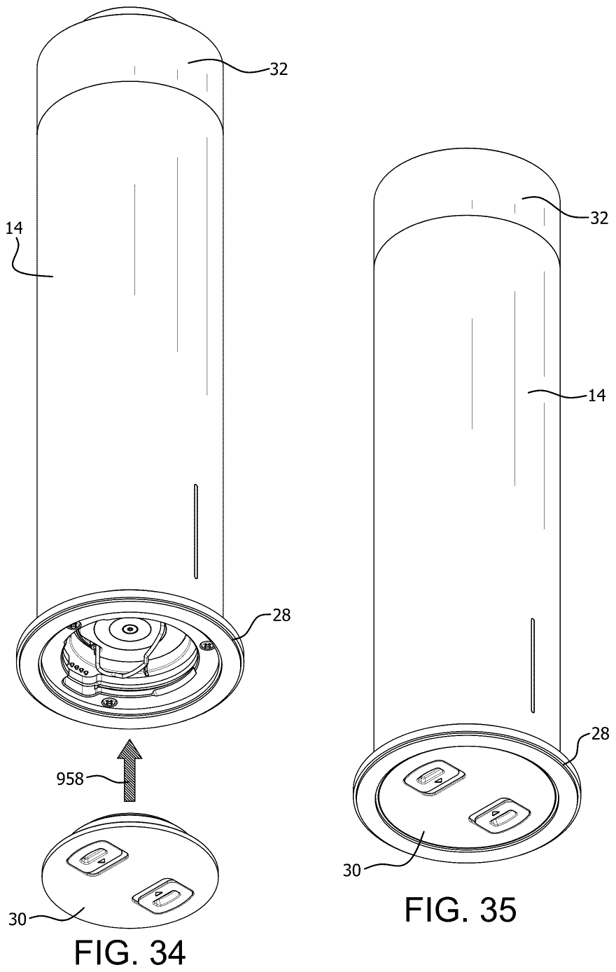

The invention allows for the replacement of the pump 45 and recycling of the pump cartridge shells 42 and 44. This will usually be performed by the manufacturer, but in some cases could be performed by the user. The pump shells 42 and 44 are dissembled by removal of the screws 508. The shells 42 and 44 are pulled apart as indicated by arrows 908 and 912 (FIG. 28-29). The electrical wires 930 and 934 are detached from contacts 940 and 942 and removed as indicated by arrow 936 (FIG. 30). A new motor 45 is replaced into the shell portion 44 as shown by arrow 938 and the electrical wires 930 and 934 are attached to the contacts 940 and 942 (FIG. 31). The shell portions 42 and 44 are then reattached as shown by arrows 950 and 952 using the pump cartridge screws 508 (FIG. 32). The cartridge 40 is then positioned into pump cartridge inside the housing 14 as shown by arrow 956 (FIG. 33). The access cover 30 is reattached as shown by arrow 958 (FIG. 34) and the scent diffuser is ready for use (FIG. 35).

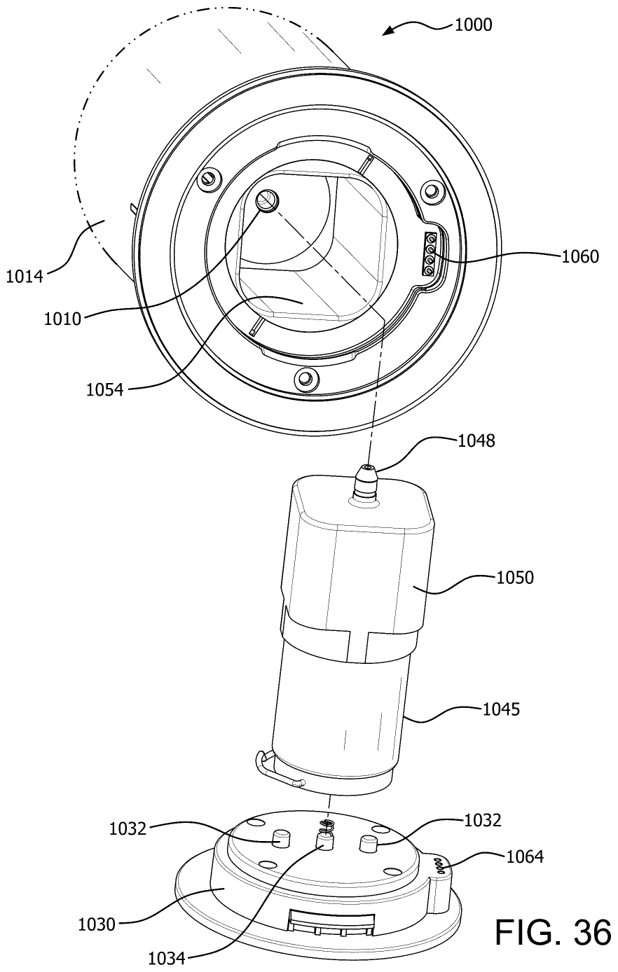

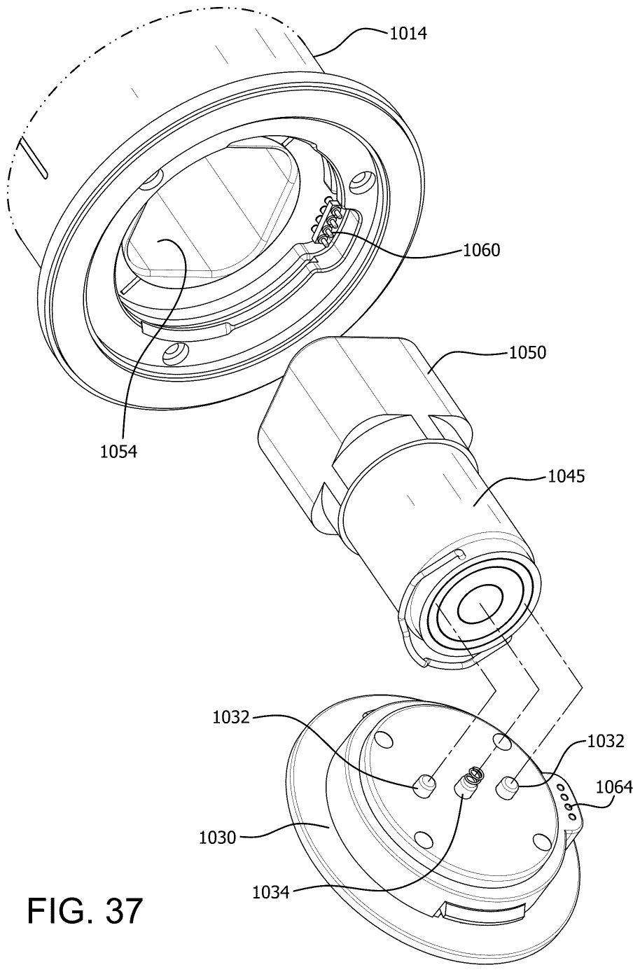

The pump cartridge 40 allows for different pumps to be used and easily replaced. In some embodiments, a cartridge is not used. This is shown in FIGS. 36-37. A diffuser housing 1014 includes an access cover 1030 with electrical contacts 1032 and 1034. A pump 1045 has a geometry such as the square top 1050, although other sizes and geometries are possible. The pump compartment 1054 is dimensioned to receive the square top 1050, such that the pump will be guided into position with the pump air outlet received by the air fitting of the pump compartment. The access cover 1030 can then be replaced, whereupon the electrical contacts 1032 and 1034 will be placed in electrical connection with pump electrical contacts 1040 and 1042. Pin contacts 1060 will be received into receptacles 1064 to deliver power from the battery or an external power source to the electrical contacts 1032 and 1034, and thereby to the pump 1045.

The invention as shown in the drawings and described in detail herein disclose arrangements of elements of particular construction and configuration for illustrating preferred embodiments of structure and method of operation of the present invention. It is to be understood however, that elements of different construction and configuration and other arrangements thereof, other than those illustrated and described may be employed in accordance with the spirit of the invention, and such changes, alternations and modifications as would occur to those skilled in the art are considered to be within the scope of this invention as broadly defined in the appended claims. In addition, it is to be understood that the phraseology and terminology employed herein are for the purpose of description and should not be regarded as limiting.

We claim:

1. A scent diffuser, comprising:
a housing having an open interior, and top, bottom and side walls for enclosing the open interior;
a reservoir container for a liquid scent;
a nozzle in fluid communication with the reservoir container, and comprising an air nozzle and a liquid nozzle, the air nozzle and liquid nozzle being arranged so as to contact an air stream flowing through the air nozzle with a liquid stream flowing through the liquid nozzle to create an atomized scent stream in the reservoir container;
a reservoir outlet in fluid communication between an atomized scent outlet in the housing and the reservoir container to release the atomized scent stream from the reservoir container into a surrounding environment;
an air pump comprising a pump air inlet and a pump air outlet;
an pump compartment having an open interior dimensioned to receive the air pump and an air fitting making a fluid connection with the pump air outlet of the air pump;
an air conduit communicating between the air fitting and the air nozzle, and a liquid conduit communicating from the reservoir container to the liquid nozzle;
the housing comprising a pump access opening and a detachable access cover for the pump access opening, the pump access opening being dimensioned to permit passage of the air pump for replacing the air pump;
a battery assembly comprising a battery and electrical conductors, the electrical conductors electrically connected with the air pump so as to power the air pump; and,
a switch assembly for controlling a flow of electric current from the battery assembly and operation of the air pump.

2. The scent diffuser of claim 1, further comprising a portable pump cartridge comprising an air pump and structure for engaging the pump compartment.

3. The scent diffuser of claim 2, wherein the structure for engaging the pump compartment is affixed to an exterior of the air pump.

4. The scent diffuser of claim 2, wherein the structure for engaging the pump compartment comprises first and second cartridge portions which when attached to each other define an open interior dimensioned to receive the air pump, and wherein the air pump is dimensioned to be received within the open interior of the pump cartridge.

5. The scent diffuser of claim 4, wherein the pump cartridge further comprises first and second electrical contacts extending from an outside surface to an interior surface of the pump cartridge, wherein when the air pump is placed within the pump cartridge electrical contacts on the pump will make electrical contact with the electrical contacts of the pump compartment.

6. The scent diffuser of claim 5, wherein one of the first and second electrical contacts on the pump cartridge is circular and the second of the first and second electrical contacts of the pump cartridge is inside of the circular electrical contact.

7. The scent diffuser of claim 3, wherein the cover to the access opening comprises first and second electrical contacts.

8. The scent diffuser of claim 1, wherein the pump air fitting and the pump air outlet comprise cooperating male and female socket connections, the male socket connection detachably mating with the female socket connection to transfer air from the air pump to the pump air fitting and thereby to the diffuser air nozzle, the female socket connection and the male socket connection permitting relative sliding attachment and detachment of the pump air outlet and the air fitting.

9. The scent diffuser of claim 8, wherein the pump air fitting is attached to the pump compartment and comprises a horizontal nipple and a sealing gasket.

10. The scent diffuser of claim 9, wherein the air conduit comprises a first air conduit extending from the horizontal nipple to a manifold, and a second air conduit extending from the manifold to the air nozzle.

11. The scent diffuser of claim 1, further comprising a status indicator for indicating at least one status characteristic selected from the group consisting of battery life, scent level in the reservoir, and pump operation.

12. The scent diffuser of claim 1, wherein the housing comprises a removable diffusion head, the diffusion head comprising the nozzle.

13. The scent diffuser of claim 12, wherein the atomized scent outlet is provided in the removable diffusion head.

14. The scent diffuser of claim 12, further comprising a tray comprising structure for engaging the reservoir container and for engaging the side walls of the housing.

15. The scent diffuser of claim 1, further comprising a check valve for preventing fluid flow through the scent outlet opening when the housing is tilted from an upright position.

16. The scent diffuser of claim 15, wherein the check valve comprises a valve member and a valve seat having a valve inlet and a valve outlet, the valve member movable to close one of the valve inlet and valve outlet to fluid flow when the housing is tilted.

17. The scent diffuser of claim 1, wherein the removable cover forms at least a part of the bottom wall of the housing.

18. The scent diffuser of claim 17, wherein the removable cover comprises a wireless charging receptor for wirelessly charging the battery.

19. A method for operating a scent diffuser, comprising the steps of:
providing a scent diffuser comprising:
a housing having an open interior, and top, bottom and side walls for enclosing the open interior;
a reservoir container for a liquid scent;
a diffuser comprising a diffuser air nozzle and a diffuser liquid nozzle in fluid communication with the reservoir container, the diffuser air nozzle and diffuser liquid nozzle being arranged so as to contact an air stream flowing through the diffuser air nozzle with a liquid stream flowing through the diffuser liquid nozzle to create an atomized air and liquid stream in the reservoir container;
a reservoir outlet in fluid communication between the reservoir container and an atomized air and liquid outlet in the housing to release the atomized air and liquid stream from the reservoir container into a surrounding environment;
an air pump comprising a pump air inlet and a pump air outlet;
a pump compartment having an open interior dimensioned to receive the air pump and an air fitting making a fluid connection with the pump air outlet of the air pump;
an air conduit communicating between the air fitting and the diffuser air nozzle, and a liquid conduit communicating from the reservoir container to the diffuser liquid nozzle;
the housing comprising a pump access opening and a detachable cover for closing the detachable cover, the pump access opening being dimensioned to permit passage of the pump through the pump access opening for replacing the air pump;
a battery assembly comprising a battery and electrical conductors, the electrical conductors communicating with the pump assembly so as to power the air pump; and,
a switch assembly for controlling the flow of electric current from battery assembly and operation of the air pump;
removing the detachable cover;
removing the air pump through the access opening; and,
replacing the air pump with a replacement air pump.
